(19) United States
(12) Patent Application Publication
(10) Pub. No.: US 2026/0253477 A1
(43) Pub. Date: Aug. 27, 2026

(54) BALANCE IMPROVEMENT DEVICE, BALANCE IMPROVEMENT METHOD, AND BALANCE IMPROVEMENT PROGRAM

(71) Applicant: NTT, Inc., Tokyo (JP)

(72) Inventors: Arinobu NIIJIMA, Musashino-shi (JP); Masato SHINDO, Musashino-shi (JP)

(73) Assignee: NTT, Inc., Tokyo (JP)

(21) Appl. No.: 19/489,636

(22) PCT Filed: Jun. 7, 2023
(86) PCT No.: PCT/JP2023/021186

§ 371 Date: Dec. 3, 2025

(51) Int. Cl. G08B 6/00; A61M 21/00; G06T 7/70

(52) U.S. Cl. CPC G08B 6/00; A61M 21/00; G06T 7/70; A61M 2021/0022; A61M 2205/3306; A61M 2230/62; G06T 2207/30196

(57) ABSTRACT

A balance improvement device is a device for reducing a fluctuation in center of gravity of a user at a standing position. The balance improvement device includes a body position detection unit, a tactile stimulus control unit, and a tactile stimulus presentation unit. The body position detection unit detects a body position of at least a part of a user's body. The tactile stimulus control unit controls a parameter for presenting a tactile stimulus to the detected body position. The tactile stimulus presentation unit presents a tactile stimulus to the detected body position in a contactless manner.

### TECHNICAL FIELD

The present invention relates to a balance improvement device, a balance improvement method, and a balance improvement program.

### BACKGROUND ART

From the viewpoint of fall prevention, it is important to improve a standing balance. As one method for improving a standing balance, a study in which a user lightly touches a wall or the like with her or his hand or fingertip to reduce a fluctuation in the center of gravity has been reported (see NPL 1). This is a phenomenon called a light touch and occurs when touching a fixed point such as a wall with a force of 1 N or less. In principle, when a situation of contact with the fixed point changes due to a fluctuation in the center of gravity, a delicate tactile receptor of the hand can sense the change and quickly sense the body's unsteadiness to restore a balance. Therefore, the fluctuation in the center of gravity is reduced.

### CITATION LIST

### Non Patent Literature

[NPL 1] John J Jeka, “Light Touch Contact as a Balance Aid”, Physical Therapy, Volume 77, Issue 5, 1 May 1997, Pages 476-487

### SUMMARY OF INVENTION

### Technical Problem

When an improvement in a standing balance is attempted using a light touch, a fixed object such as a physical wall or a handrail is necessary. Accordingly, in a place where there is no fixed object such as a wall or a handrail, a standing balance cannot be improved using a light touch. When a handrail is additionally installed, there is also an adverse effect such as spoiling the landscape or narrowness of a road width. Further, due to being a fixed object, it is also difficult to change a height according to a user's height.

The present invention has been made in view of the above circumstances, and the object of the present invention is to provide a balance improvement device, a balance improvement method, and a balance improvement program for providing an improvement in a standing balance using a light touch without requiring a fixed object.

### Solution to Problem

An aspect of the present invention is a balance improvement device for reducing a fluctuation in the center of gravity of a user at a standing position. The balance improvement device includes a body position detection unit, a tactile stimulus control unit, and a tactile stimulus presentation unit. The body position detection unit detects a body position of at least a part of a user's body. The tactile stimulus control unit controls parameters for presenting a tactile stimulus to the detected body position. The tactile stimulus presentation unit presents a tactile stimulus to the detected body position in a contactless manner.

Another aspect of the present invention is a balance improvement method for improving a balance to reduce a fluctuation in the center of gravity of a user at a standing position. The balance improvement method includes: a step of detecting a body position of at least a part of the user's body; a step of controlling a parameter for presenting a tactile stimulus to the detected body position; and a step of presenting a tactile stimulus to the detected body position in a contactless manner.

Still another aspect of the present invention is a balance improvement program for reducing a fluctuation in the center of gravity of a user at a standing position. A balance improvement program causes a computer that includes a processor and a storage device to perform at least part of control performed by a tactile stimulus control unit of the balance improvement device.

### Advantageous Effects of Invention

The present invention provides a balance improvement device, a balance improvement method, and a balance improvement program for providing an improvement in a standing balance using a light touch without requiring a fixed object.

### BRIEF DESCRIPTION OF DRAWINGS

FIG. 1 is a block diagram illustrating a functional configuration of a balance improvement device according to an embodiment.

FIG. 2 is a flowchart illustrating a flow of processing performed by the balance improvement device according to the embodiment.

FIG. 3 is a block diagram illustrating a functional configuration of the balance improvement device in an implementation example.

FIG. 4 is a flowchart illustrating a flow of processing performed by the balance improvement device in an implementation example.

FIG. 5 is a diagram illustrating an experiment result of reducing a fluctuation in the center of gravity when subjects performed a standing test on both feet for 30 seconds while their eyes were closed.

FIG. 6 is a block diagram illustrating a hardware configuration of a computer configuring a tactile stimulus control unit of the balance improvement device.

### DESCRIPTION OF EMBODIMENTS

Hereinafter, embodiments according to the present invention will be described with reference to the drawings.

### Functional Configuration

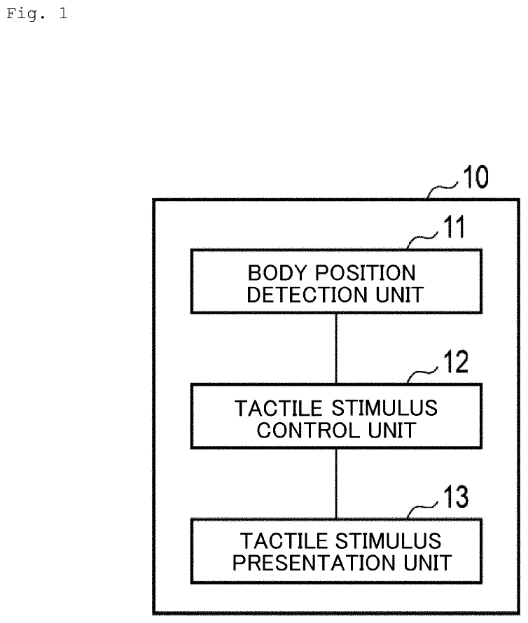

First, a balance improvement device 10 according to an embodiment will be described with reference to FIG. 1. FIG. 1 is a block diagram illustrating a functional configuration of the balance improvement device 10 according to an embodiment. The balance improvement device 10 is a device for reducing a fluctuation in the center of gravity of a user at a standing position.

The balance improvement device 10 includes a body position detection unit 11, a tactile stimulus control unit 12, and a tactile stimulus presentation unit 13. The body position detection unit 11 detects a body position of at least a part of a user's body. The tactile stimulus control unit 12 controls a parameter of the tactile stimulus presentation unit 13 so that the tactile stimulus is presented to the body position detected by the body position detection unit 11. The tactile stimulus presentation unit 13 localizes a tactile stimulus at the body position detected by the body position detection unit 11 and presents the tactile stimulus in a contactless manner by operating according to the parameters controlled by the tactile stimulus control unit 12.

### Example of Operations

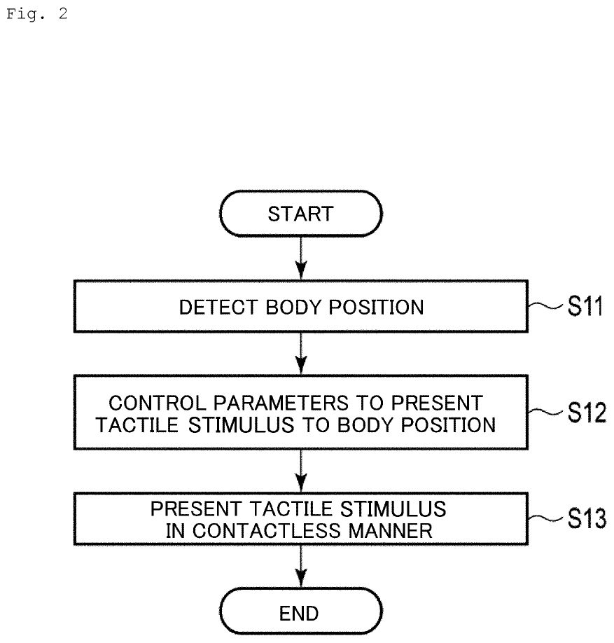

Next, processing for improving a balance performed by the balance improvement device 10 will be described with reference to FIG. 2. FIG. 2 is a flowchart illustrating a flow of balance improvement processing performed by the balance improvement device 10 according to an embodiment.

In step S11, the body position detection unit 11 detects a body position of at least a part of the user's body. The body position detection unit 11 outputs information regarding the detected body position to the tactile stimulus control unit 12.

In step S12, the tactile stimulus control unit 12 controls the parameter of the tactile stimulus presentation unit 13 such that the tactile stimulus is presented to the body position based on the information regarding the body position input from the body position detection unit 11.

In step S13, the tactile stimulus presentation unit 13 operates in accordance with the parameter controlled by the tactile stimulus control unit 12 to localize the tactile stimulus at the body position detected by the body position detection unit 11 and present the tactile stimulus in a contactless manner.

### Implementation Example

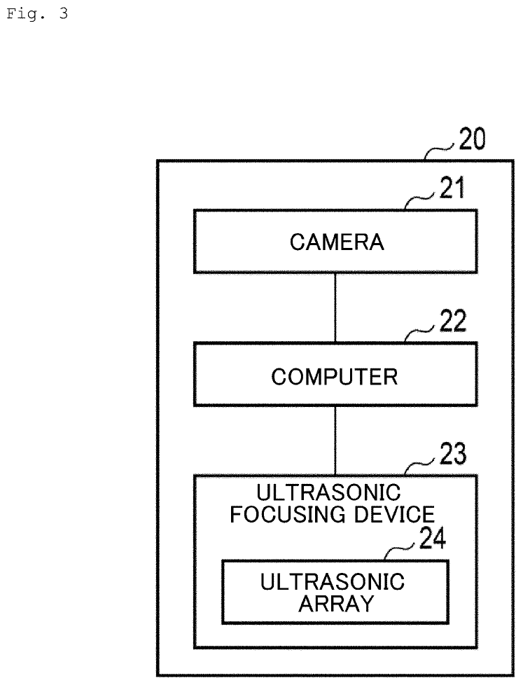

Next, an implementation example of the balance improvement device 10 will be described with reference to FIG. 3. FIG. 3 is a block diagram illustrating a functional configuration of the balance improvement device 20 according to an implementation example.

The balance improvement device 20 includes a camera 21 configuring the body position detection unit 11, a computer 22 configuring the tactile stimulus control unit 12, and an ultrasonic focusing device 23 configuring the tactile stimulus presentation unit 13.

The camera 21 has a function of detecting a position of part of a subject. For example, techniques for artificial intelligence (AI) and computer vision are incorporated in the camera 21. Accordingly, the camera 21 can detect a position of each part of the imaged subject, for example, a user's hand. The camera 21 outputs information regarding the detected position of the user's hand to the computer 22.

The ultrasonic focusing device 23 radiates an ultrasonic wave as a tactile stimulus. The ultrasonic focusing device 23 includes an ultrasonic array 24. The ultrasonic array 24 is a structure in which a plurality of ultrasonic transducers are arrayed. For example, the ultrasonic focusing device 23 can focus ultrasound waves generated from the ultrasonic array 24 by phase control. The ultrasonic focusing device 23 can also adjust power of the ultrasonic wave generated from the ultrasonic array 24 by amplitude control.

The computer 22 is, for example, a personal computer (PC). The computer 22 controls parameters of the ultrasonic focusing device 23 so that the ultrasonic wave emitted from the ultrasonic focusing device 23 is focused on the position of the user's hand based on the information of the position of the user's hand input from the camera 21. For example, the computer 22 focuses the ultrasonic wave emitted from the ultrasonic focusing device 23 on the position of the user's hand by controlling a parameter related to a phase of the ultrasonic wave generated from the ultrasonic array 24. The computer 22 also controls a parameter related to amplitude of the ultrasonic wave generated from the ultrasonic array 24 such that the ultrasonic wave focused on the position of the user's hand is presented to the user as a weak tactile feedback of 1 N or less. The tactile feedback is a stimulation that causes the user to feel a tactile sensation when the user touches a fixed object.

Under the control of the computer 22, the ultrasonic wave emitted from the ultrasonic focusing device 23 is focused on one point of the position of the user's hand and provides a weak tactile feedback of 1 N or less to the user. The weak tactile feedback presented to one point on a fingertip or a palm gives the user a sensation similar to touching a fixed object such as a wall with a hand or a fingertip. When the hand moves due to a fluctuation in the center of gravity, a method of touching a localized point of the ultrasonic wave is changed and the change can be perceived by a tactile receiver of the hand. Therefore, the user can reduce a fluctuation in the center of gravity by quickly recovering the balance. That is, the user can improve the standing balance by using a light touch.

### Operation Example

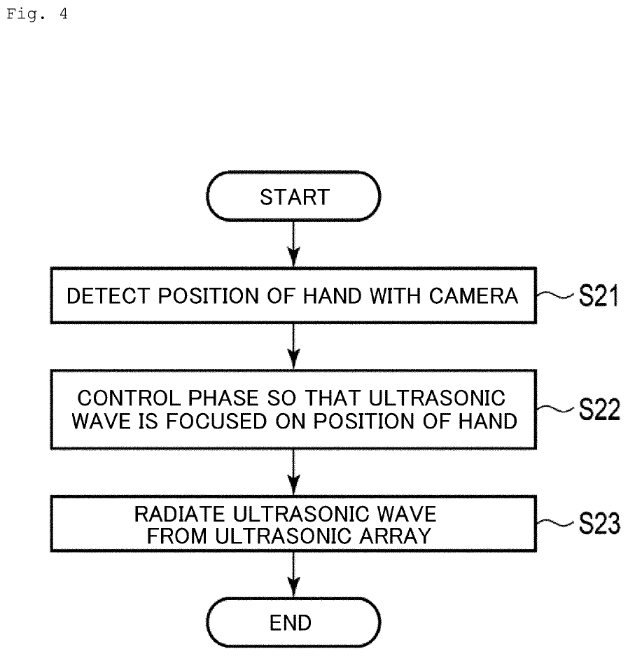

Next, balance improvement processing performed by the balance improvement device 20 will be described with reference to FIG. 4. FIG. 4 is a flowchart illustrating a flow of balance improvement processing performed by the balance improvement device 20.

In step S21, the balance improvement device 20 detects a position of the user's hand with the camera 21. The camera 21 outputs information regarding the detected position of the user's hand to the computer 22.

In step S22, the computer 22 controls a parameter related to the phase of the ultrasonic wave generated from the ultrasonic array 24 such that the ultrasonic wave emitted from the ultrasonic focusing device 23 is focused on one point of the position of the user's hand based on the information regarding the position of the user's hand input from the camera 21. The computer 22 also controls a parameter related to the amplitude of the ultrasonic wave generated from the ultrasonic array 24 such that the ultrasonic wave focused on the position of the user's hand presents a weak tactile feedback of 1 N or less to the user.

In step S23, the ultrasonic focusing device 23 operates in accordance with the parameter controlled by the computer 22 and radiates the ultrasonic wave from the ultrasonic array 24. The ultrasonic wave emitted from the ultrasonic array 24 is focused on one point of the position of the user's hand and present a weak tactile feedback of 1 N or less to the user.

The user receiving the weak tactile feedback can use a light touch to reduce the fluctuation in the center of gravity and improve a standing balance.

### Experimental Example

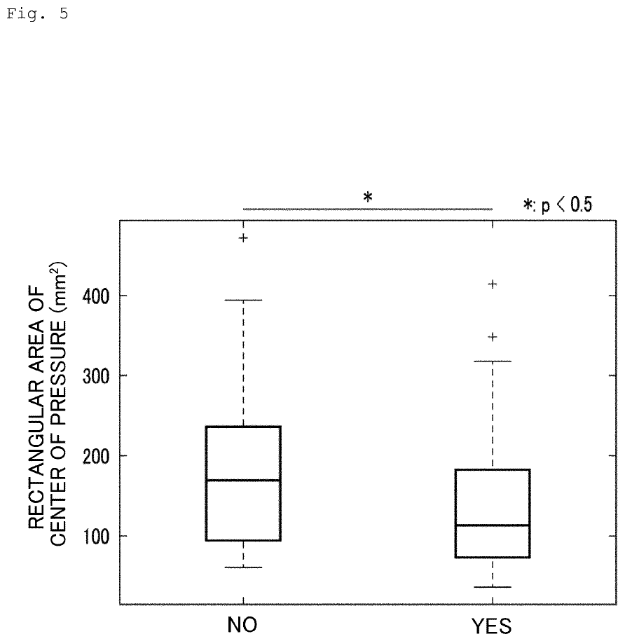

An experimental example in which an experiment was actually conducted for twenty three subjects aged 20 to 50 years old who performed a 30-second closed-eyes standing test on both feet will be described with reference to FIG. 5. FIG. 5 is a diagram illustrating an experimental result of reducing a fluctuation in the center of gravity when twenty three subjects aged 20 to 50 years old performed a 30-second closed-eyes standing test on both feet. In the experimental example, a force plate was used to measure a center of a foot pressure position, and a rectangular area of a movement range for 30 seconds was calculated for each subject. Two conditions of a case of measurement without ultrasonic stimulation and a case of measurement with an ultrasonic stimulation on a palm were compared. When median values are compared in each condition, the rectangular area was 170 mm 2 in absence of the ultrasonic wave and the rectangular area was 113 mm 2 in the presence of the ultrasonic wave. With an ultrasonic stimulation, a 34% reduction in the rectangular area is shown. It can be said that the smaller the rectangular area is, the less the fluctuation in the center of gravity is and the more the balance is stable, and thus it can be said that an effect of improving a standing balance is obtained according to the embodiment.

### Hardware Configuration

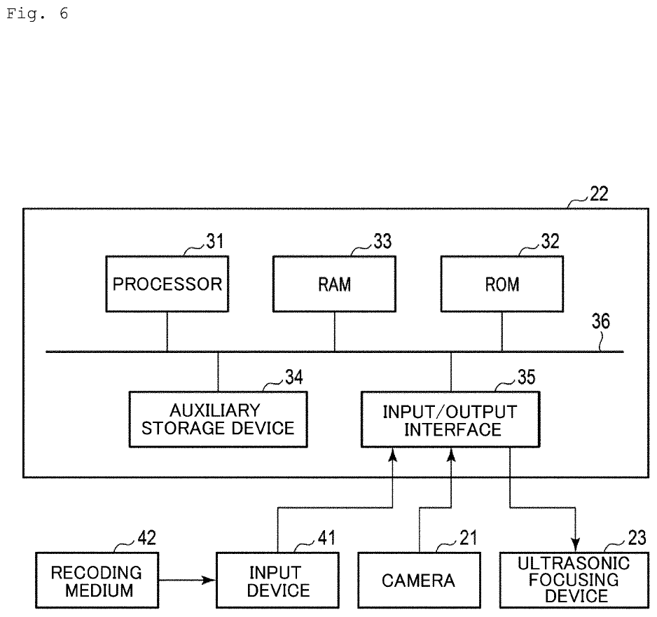

Next, a hardware configuration of the computer 22 configuring the tactile stimulus control unit 12 of the balance improvement device 10 will be described with reference to FIG. 6. FIG. 6 is a block diagram illustrating a hardware configuration of the computer 22 that configures the tactile stimulus control unit 12 of the balance improvement device 10.

The computer 22 includes a processor 31, a read only memory (ROM) 32, a random access memory (RAM) 33, an auxiliary storage device 34, and an input/output interface 35.

The processor 31, the ROM 32, the RAM 33, the auxiliary storage device 34, and the input/output interface 35 are electrically connected to each other via a bus 36, and perform transmission and reception of data via the bus 36.

The processor 31 is configured with a general-purpose hardware processor including, for example, a central processing unit (CPU) and a graphical processing unit (GPU). The processor 31 controls all the ROM 32, the RAM 33, the auxiliary storage device 34, and the input/output interface 35.

The ROM 32 is a nonvolatile memory that configures a part of a main storage device. The ROM 32 non-temporarily stores a starting program required when the processor 31 starts. The processor 31 is started by executing a program in the ROM 32. The ROM 32 is configured with, for example, an erasable programmable read only memory (EPROM) and stores settings at the time of starting in addition to a startup program.

The RAM 33 is a volatile memory configuring a part of the main storage device. The RAM 33 temporarily stores a program necessary for processing of the processor 31 and data necessary for executing the program. The processor 31 calculates data in the RAM 33 by executing a program in the RAM 33 and stores a calculation result in the RAM 33.

The auxiliary storage device 34 is configured with a nonvolatile memory such as a hard disk drive (HDD) or a solid state drive (SSD). The auxiliary storage device 34 non-temporarily stores a program executed by the processor 31 and data necessary for executing the program. The processor 31 reads the program and data in the auxiliary storage device 34 to the RAM 33 and executes the program to execute various functions.

The input/output interface 35 is connected to the camera 21, the ultrasonic focusing device 23, the input device 41, and the like so that information can be input and output. For example, the input/output interface 35 may be a wired interface or a wireless interface. The wired interface includes a port to which a device is connected. The wireless interface includes Bluetooth (registered Trademark), and WiFi (registered trademark).

The input device 41 can include a keyboard, a mouse, a touch panel, a reception device, and a disk drive. The input device 41 is not limited thereto and may include any other input devices.

The program non-temporarily stored in the auxiliary storage device 34 is supplied to the computer 22 via, for example, a recording medium 42 which is readable by the computer 22 recording the program non-temporarily. The recording medium 42 is called a non-transitory computer-readable recording medium. Examples of non-transitory computer-readable recording media include disks such as flexible disks, optical discs (CD-ROM, CD-R, DVD-ROM, DVD-R, and the like), magneto-optical discs (MO and the like), and semiconductor memories.

The programs non-temporarily stored in the auxiliary storage device 34 include a balance improvement program. The balance improvement program causes the computer 22 to execute at least some of the functions of the tactile stimulus control unit 12.

The program non-temporarily stored in the auxiliary storage device 34 is read and non-temporarily stored in, for example, the auxiliary storage device 34 via a disk drive, which is the input device 41, and the input/output interface 35 when the recording medium 42 is a disk. The program may be stored in a server on a network, downloaded from the server, and non-temporarily stored in the auxiliary storage device 34.

The processor 31 executes a program in the ROM 32 when the computer 22 is started, reads an OS to the RAM 33 and starts the OS. The processor 31 monitors an instruction input, connection of an external device, or the like under the control of the OS. The processor 31 sets a program area and a data area in the RAM 33 under control of the OS. In response to an instruction input to start the tactile stimulus control unit 12, the processor 31 reads the balance improvement program to the program area of the RAM 33 from the auxiliary storage device 34, and also reads data required for executing the balance improvement program from the auxiliary storage device 34 to the data area of the RAM 33. The processor 31 calculates data of the data area according to the balance improvement program and writes a calculation result in the data area. Through such an operation, the processor 31, the RAM 33, the auxiliary storage device 34, the input/output interface 35, and the bus 36 cooperate to execute at least some of the functions of the tactile stimulus control unit 12.

### Effects

According to an embodiment, even in a place where there is no fixed object such as a wall or a handrail, an improvement in standing balance using a light touch can be provided to the user. Since the ultrasonic wave is not visible and can be radiated from a remote place, there is no adverse effect such as damage of landscape and narrowness of a road width.

Further, because the position of a convergence point of the ultrasonic wave can be changed by controlling the ultrasonic array, a tactile feedback can be presented to any height to match a user's height. That is, it is possible to provide a technique for providing an improvement in standing balance using a light touch without the need for a fixed object.

### Modification Example

In addition to the camera 21, the body position detection unit 11 may be configured using an infrared camera, an ultrasonic sensor, a magnetic sensor, a laser distance sensor, a motion capture, and a depth sensor.

In addition to the ultrasonic focusing device 23, the tactile stimulus presentation unit 13 may be configured using a laser device, an air jet, a directional speaker, and a temperature stimulation presentation device.

In any case, a position of a part of the user's body may be detected and the tactile stimulus may be presented to be localized at that position.

A plurality of sets of cameras 21 and the ultrasonic focusing device 23 may be included to present tactile stimuli over a wide range.

Further, as the determination of whether to present a tactile stimulus, a function of presenting a tactile stimulus only when the body position detection unit 11 determines that a fluctuation in the center of gravity is equal to or more than a given value may be added.

A part to which the tactile stimulus is presented is not limited to a hand, but may be any part on the body such as a head, a back, a foot, or the like.

Instead of focusing the ultrasonic wave by phase control, an acoustic lens or the like may be used to focus the ultrasonic wave. In this case, the computer 22 controls a parameter such as an acoustic lens.

The present invention is not limited to the above-described embodiments and various modifications can be made in an implementation stage without departing from the gist of the invention. The embodiments may be combined as appropriate.

In that case, the combined effects can be obtained. Further, the foregoing embodiments include various inventions and various inventions can be extracted by combinations selected from the plurality of disclosed constituent elements. For example, even when some of the constituent elements are deleted from all the constituent elements described in the embodiments, the configuration from which these constituent elements are deleted can be extracted as an invention as long as the problem can be solved and the effects can be obtained.

### REFERENCE SIGNS LIST

10 Balance improvement device
11 Body position detection unit
12 Tactile stimulus control unit
13 Tactile stimulus presentation unit
20 Balance improvement device
21 Camera
22 Computer
23 Ultrasonic focusing device
24 Ultrasonic array
31 Processor
32 ROM
33 RAM
34 Auxiliary storage device
35 Input/output interface
36 Bus
41 Input device
42 Recording medium

1. A balance improvement device for reducing a fluctuation in center of gravity of a user at a standing position, the balance improvement device comprising processing circuity configured to:
detect a body position of at least a part of a body of the user;
control a parameter for presenting a tactile stimulus to the body position that has been detected; and
present a tactile stimulus to the body position that has been detected in a contactless manner.

2. The balance improvement device according to claim 1,
wherein the processing circuity is configured with a camera having a function of detecting a position of each part of a subject,
wherein the processing circuity is configured with an ultrasonic focusing device that emits an ultrasonic wave, and
wherein the processing circuity is configured with a computer that controls the ultrasonic focusing device.

3. The balance improvement device according to claim 2,
wherein at least the part of the body of the user is a hand,
wherein the computer controls a parameter of the ultrasonic focusing device to focus the ultrasonic wave emitted from the ultrasonic focusing device at a position of the hand.

4. The balance improvement device of claim 3, wherein the computer controls a parameter of the ultrasonic focusing device to present a tactile feedback of 1 N or less to the user by the ultrasonic wave focused to the position of the hand.

5. The balance improvement device according to claim 2, wherein the computer controls a phase of the ultrasonic wave emitted from the ultrasonic focusing device to focus the ultrasonic wave.

6. The balance improvement device according to claim 2, further comprising a plurality of sets of a camera and an ultrasonic focusing device each including the camera and the ultrasonic focusing device to present a tactile stimulus of ultrasonic wave over a wide range.

7. A balance improvement method for reducing a fluctuation in center of gravity of a user at a standing position, the method comprising:
detecting a body position of at least a part of the body of the user;
controlling a parameter for presenting a tactile stimulus to the body position that has been detected; and
presenting a tactile stimulus to the body position that has been detected in a contactless manner.

8. A non-transitory storage medium storing at least one program causing a computer that includes processing circuity and a storage device to perform the steps of:
detecting a body position of at least a part of the body of the user;
controlling a parameter for presenting a tactile stimulus to the body position that has been detected; and
presenting a tactile stimulus to the body position that has been detected in a contactless manner.
